(19) United States
(12) Patent Application Publication
(10) Pub. No.: US 2019/0351002 A1
(43) Pub. Date: Nov. 21, 2019

(54) METHOD OF TREATING CANCER, PARASITE, MICROBIAL OR FUNGAL DISEASE

(71) Applicant: Baris DUZGUN, Samsun (TR)

(72) Inventor: Baris DUZGUN, Samsun (TR)

(21) Appl. No.: 16/525,842

(22) Filed: Jul. 30, 2019

Related U.S. Application Data
(62) Division of application No. 15/539,138, filed on Jun. 22, 2017, now abandoned.

(51) Int. Cl. A61K 36/9068; A61K 9/107; A61K 9/10; A61K 47/20

(52) U.S. Cl. CPC A61K 36/9068; A61K 9/107; Y02A 50/473; A61K 47/20; A61K 9/10

(57) ABSTRACT

A method of treating cancer, parasite, microbial or fungal disease, comprising administering a Ginger (Zingiber officinale) extract solution to treat the cancer, parasite, microbial or fungal disease; wherein the Ginger (Zingiber officinale) extract solution comprises extracts of Ginger (Zingiber officinale), and a DMSO (dimethyl sulphoxide) solvent; and wherein the extracts of Ginger (Zingiber officinale) are obtained by cold pressing; and an effective dose of the extracts of Ginger (Zingiber officinale) is between a range of 25 μg/ml-12800 μg/ml, and a content of the DMSO (dimethyl sulphoxide) solvent is between a range of 0.1-2 wt %; and an anti-parasite effective dose of the extracts of Ginger (Zingiber officinale) on Toxoplasma gondii is 50 μg/ml-3200 μg/ml, and the content of the DMSO (dimethyl sulphoxide) solvent is 0.1 wt %.

### CROSS REFERENCE TO RELATED APPLICATIONS

This application is a divisional application of application Ser. No. 15/539,138 filed on Jun. 22, 2017, which is the national phase entry of International Application No. PCT/TR2015/000264, filed on Jun. 18, 2015, which is based upon and claims priority to Turkey Patent Application No. 2014/15529, filed on Dec. 22, 2014, the entire contents of which are incorporated herein by reference.

### TECHNICAL FIELD

The invention is related to particularly the antineoplastic (anticancer), anti-parasite and antimicrobial efficiency of the solution formed of Ginger extract. The invention is related to the field of Medicine and Biology. An effective dose on the neoplastic tissue cells with the emulsion formed of ginger plant following the study conducted on Human Larynx Epidermoid cancer (Hep2) cells has been found and a formulation having antimicrobial and anti-parasite efficiency in some microorganisms that shall be given with detail below according to additional studies carried out has been provided.

### BACKGROUND

Various studies are being carried out nowadays regarding human health. In such studies, developments are observed in many fields, particularly in human and environmental health and inventions are conducted which can be an alternative to or replace present modalities.

Cancerous structures, are formed of cells and tissue masses which are broken away from normal life cycle and which generally increase excessively, and which disseminate in the related areas in which they have increased or if they have shown metastasis. Control mechanism disorders are generally frequently observed in genetic activities of pathognomonic mechanisms and these types of cells. There are many types of cancerous cells such as epithelial, mesenchymal, squamosal, adenocarcinoma etc. according to tissue and cell groups from which cancer cells are originated. Cancer cells and tissue types define the efficiency of many different chemotherapeutic agents. Chemotherapeutic agents try to prevent the increase and to inactivate the cancerous cells by affecting the metabolic pathways of cancerous tissue cells. During this treatment, sometimes healthy cells are also damaged due to tissue deterioration in connection with chemotherapeutic agents.

Toxoplasmosis which has a normal progress of 90% in general in individuals having a strong immune system shows severe progress in cases of diseases such as AIDS which causes immunity disorders particularly dependent on T cells, haematological cancers, bone marrow and solitary organ transplants and if this progress is not controlled the results can be malignant.

The consumption of foods that have not been cooked properly may lead to cysts, consuming food that contains oocysts, transplacental tissue and organ transplants, laboratory accidents, mechanic vector borne infections from invertebrate animals may be counted as ways of transmission of infections.

Although medicines such as pyrimethamine and sulphadiazine are used, it is impossible to completely eliminate the factor that causes the disease. Age groups, especially childhood also restrict medicine usage.

The struggle against inflammatory diseases where gram positive and gram negative microorganisms are effective such as parasitic diseases, constitutes an important problem nowadays and either tolerances are developed against many drugs that are being sold in the market by each passing day, or cases are observed where patients do not respond to treatment. High generation drug usage in microbial diseases causes the creation of different resistance profiles, increases the treatment costs, extends the time of stay in the hospital if the patient is an inpatient, and may lead to additional complications. Due to these reasons, the health of the individual or the community is either directly or indirectly affected and life comfort is disturbed. Many antibiotics from a similar group or having different chemical structures have been put on the market since the discovery of penicillin group antibiotics. However together with each drug, resistance cases have also started to be observed.

Efficient treatment regimes with minimum adverse affects to human health regarding infections of protozoons and other parasitic infections primarily T. gondii, have still not exactly been found.

Similar problems are faced during the usage of antimicrobial drugs used to treat parasitic infections and infectious diseases; either the usage range is restricted due to their adverse affects and problems are especially faced in relation to age groups; or insufficiencies are observed in the fight against many diseases for which an effective treatment has not been discovered yet.

Nowadays there are a lot of cancer patients. The most significant part of the treatment protocols in the struggle against cancer is still early diagnosis and this also determins the limits of both drug treatment and surgical operation opportunities. In late diagnosis many antineoplastic drugs also cause severe damage to the body; moreover many patients are lost because of complications.

Cancerous structures are usually formed of cells which do not increase normally; which have broken away from the life cycle and can be severely aggressive. These type of cells, may cause excessive cellular activity, migration to different organs and tissues, creation of masses, dependant complications, tissue deterioration etc. They may cause dysfunctions in the organs and tissues, balance and functional disorders, and organ failures. The patients have difficulty continuing their lives as normal due to the above mentioned or other similar reasons.

The development of alternative drugs which show high efficiency against tumor cells, while having minimal effect on healthy cells can be a solution to this great problem.

During the study, tissue culture modelling in vitro has been carried out, thereby imitating living tissues. Cytopathic doses in cancerous serial cells related to a study formed of several components and the efficient doses of vegetal extracts have been calculated in terms of mg/ml. As a result of the studies carried out by taking cytopathic dose as basis, treatment efficiency has been discovered on the protozoon named Toxoplasma gondii. The doses that are found by means of the study carried out simultaneously with reference drug molecules shall be able to be applied to living beings.

It has been discovered that the ginger plant has antimicrobial effect on Gram positive and Gram negative microorganisms in coordination with cancer and anti-parasitic efficiency.

### SUMMARY

Cancerous cells are formed as a result of excessive out of control division of cells with various pathogenic mechanisms or as a result of processes arising due to the disruption of mechanisms which suppress such processes.

Toxoplasma gondii is a protozoon parasite. Although treatment options are restricted, a 100% efficient treatment modality has not been discovered today and reactivations can be observed, depending on immunity.

Vegetal extracts constitute the principle aspect of alternative and modern medicine. The extract of Ginger (Zingiber officinale) obtained via cold pressing has been used in our study. 100% pure seed oil has not been found to be suitable for direct usage during studies. The pure extract obtained from the plant is diluted with various diluents and solvents; thereby obtaining forms which can infuse into cells and can absorb other connected contents. Various steps need to be taken in order to be able to conduct a study regarding its efficiency in human beings.

According to the study subject to the invention;

1. The effective concentration of Zingiber officinale on cancerous cell line has been determined as EC50.

2. The effective dose and doses in terms of mg/ml has been found against the active agents of the medicines used in treating with pyrimethamine and sulphadiazine on Toxoplasma gondii.

3. It has also been discovered that Zingiber officinale had a lethal effect on various microorganisms.

### BRIEF DESCRIPTION OF THE DRAWINGS

FIG. 1: Table showing the results obtained from the MTT test when DMSO has been used as a solvent;

FIG. 2: Table showing the various concentrations in the MTT test of Ginger;

FIG. 3: Table showing the section view of a plate used in ELISA testing of Ginger.

### DETAILED DESCRIPTION

Ginger (Zingiber officinale) is a plant of the Zingiber family which can reach a height of on meters, having thin long leaves, and yellow and red flower blossoms. It has perennial tuberous or rhizome roots. It forms an annual stem (artificial body) having dark green leaves. The flower cluster rises up from a small stem from the root.

Important active ingredients can be found in the composition of ginger roots. Fresh ginger comprises 80% water, 2% protein, 1% oil, 12% yeast, calcium, phosphor, iron, B and C vitamins.

The bradyzoite formed within the cyst that enables the protection of the toxoplasma parasite from the host immune system and causes the recurrence of the disease; thereby this makes controlling and eradication of the disease difficult. Although drugs are used for treatment it is not possible to completely eliminate the factor causing the disease. The side effects in connection with the disease also cause a significant problem.

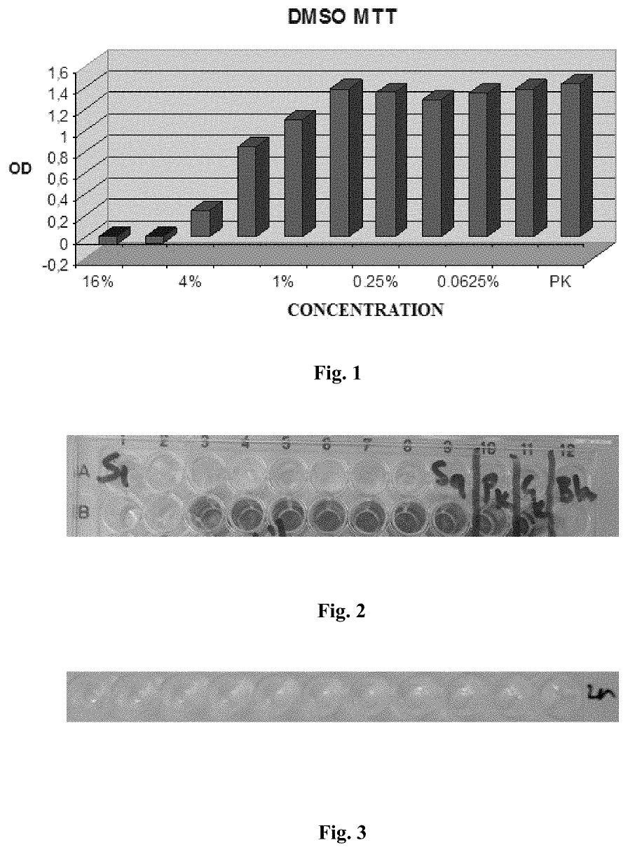

Nowadays where alternative medicine has gained importance day by day, the discovery of antimicrobial, anti-parasite agents not only play an important role in terms of toxoplasmosis but also in general diseases. Pyrimethamine and sulphadiazine have been taken as basis as reference drugs, and have been compared with vegetal extracts in vitro. Hep2 (Human Epidermoid Larynx ca) cells (FIG. 1) which is a cancer cell line is used as basis in our study.

The oils of the plant as an extract are used which have been obtained by cold pressing and it has been ensured that the extract is fresh and pure. The extracts that have been obtained through the whole study have been initially diluted with solvents, filtered and sterilized. Cytotoxicity study (MTT test) has been developed as a method regarding the Hep2 cell line in all parameters for initial doses. The maximum dose and dilution sub doses that have been determined accordingly have been repeated controlled groups.

Different amounts of Hep2 cells have been tested in flat bottomed 96 well plates for cell culture during the first stage of the study and it has been determined that 2×10⁴ cells were optimal for the monolayer layer of 2×10⁴ cell per well.

Different Toxoplasma gondii tachyzoite suspensions have been formed during the determination of the dose which could infect the cells within the cell culture plate. The highest rate was determined to be 2×10⁶/ml. During evaluations carried out with an invert microscope, cell deteriorations were quite high in high T. gondii amounts. The suspension containing 6×10⁴/ml tachyzoite has been determined to be the most suitable amount in MTT testing (Dimethylthiazole-Diphenyl Tetrazolium Bromide) cytopathically.

The optical density (OD) of the control cell group has been found to be 1.764, whereas the tachyzoites per number were respectfully determined in average as follows for 2×10⁶/ml; 0.094, 10⁶/ml; 0.096, 5×10⁵/ml; 0.097, 2.5×10⁵/ml; 0.357, 1.25×10⁵/ml; 0.651, 6×10⁴/ml; 0.864, 3×10⁴/ml; 1.238, 1.5×10⁴/ml; 1.575, and for 7×10³/ml; 1.438. According to the OD of the control group, the OD of the 6×10⁴/ml tachyzoite loaded group was determined to be 0.864; and it has been determined to be the most suitable tachyzoite number for the study according to the cell deterioration observed on the plate surface, as a tachyzoit number and according to the control group.

A pure oil of Zingiber officinale extract, which has been tested inside various solvents in terms of penetration into cells and homogenous distribution. MTT tests have been carried out relating to Hep2 cells in solvents too. DMSO was selected from solvents such as DMSO and Methanol and was used and cytopathic doses have not been exceeded during the whole study (FIG. 1). Essential oil has been obtained from Ginger and emulsions have been obtained from fixed oil plants (Nigella Sativa etc.) and compounds.

According to MTT test (FIG. 2) results that belong to Zingiber officinale, the average OD values of the control group have been determined as 1.38. While the OD value of 800 μg/ml ginger extract was found to be 0.67, gradual increase in the OD values that are observed were as follows; in 400 μg/ml: 1.08, in 200 μg/ml concentration 1.16, in 100 μg/ml 1.35, in 50 μg/ml: 1.39 etc. The suitable EC₅₀ value has been determined as 800 μg/ml.

Ginger extract solution doses of 1600 μg/ml and above (1600 μg/ml-12800 μg/ml) have also been studied on and tested. Similar anti-cancerous, anti-parasite, and antimicrobial efficiencies have also been observed in these doses. During the study the content of DMSO has been kept at 0.1%, but during the above mentioned doses, the DMSO content was increased at a range between 0.1-2% in order to increase the solubility of Ginger.

8 different concentrations have been diluted and tested in the MTT test carried out on Pyrimethamine and the below mentioned results have been obtained. The OD value of the highest concentration of pyrimethamine which is 32 μg/ml was determined to be 0.097. The OD values gradually increased as the dosage increased as follows: 0.209 in 16 μg/ml, 0.310 in 8 μg/ml, 0.623 in 4 μg/ml, 1.198 at a 2 μg/ml concentration and 1.228 in 1 μg/ml. Doses around 4 μg/ml and below have been used in the control group.

The OD value of 200 μg/ml which is the highest concentration in sulphadiazine was determined to be 0.254. The OD values which showed gradual increase according to dosage have been determined to be 0.827 in 100 μg/ml and 1.002 in 50 μg/ml concentrations.

An ELISA (Enzyme-Linked Immunosorbent Assay) test (FIG. 3) has been planned and conducted in order to determine efficiency against Toxoplasma gondii. The plant and drug doses determined during MTT tests have been studied on as multiple groups and their efficiencies have been compared with each other.

The ELISA test has been carried out on flat bottom well plates having 96 wells. First of all, the bases of the plates have been covered with a suitable amount of Hep2 cells. They have been left to be infected with Toxoplasma gondii cells for 4 hours, except the negative control group and following this elution has been carried out and the supernatant has been removed. Following this all wells have been processed again with different doses (determined with MTT) of pyrimtheamine, sulphadiazine—and Ginger (Zingiber officinals) extract, prepared with DMSO. The below mentioned study protocol has been applied in order to determin the efficiency against T. gondii:
The Hep2 cell suspension has been freshly prepared one day before according to the study protocol that has been established.
Counting has been made on Thome slide, and the vitality rate was monitored after staining with Trypan Blue.
2×10⁴ Hep2 cells were used for each well of the ELISA plate.
The negative control wells have been processed with only cell and medium during the whole of the test processes.
Cells have been provided freshly from 25′ or 75′ flasks.
Cells have been incubated for 24 hours in a 5% CO₂ incubator at +37° C.
The plate medium which has become monolayered and confluent has been emptied and washed with PBS.
EC50 has been targeted according to the MTT viability test results of medicines and the initial doses for an ELISA test were as follows:

| | |
|---|---|
| Pyrimethamine | 4 μg/ml |
| Sulphadiazine | 100 μg/ml |

6×10⁴/ml solution of T. gondii tachyzoites for an ELISA plate has been established.
The plate that was washed with PBS, was processed with a 100 μl tachyzoite suspension except for NK (negative control) and has been incubated for a minimum of 4 hours.
Following this the wells were emptied and were pipetted with 200 μl PBS.
The solutions which belong to drug and extracts that have been formed by dilution and 5% EMEM medium were loaded as dilutions.
Two different ELISA plates were prepared which belong to two different periods of time being 48 and 72 hours.
Each parameter was repeated at least 3 times in order to increase the efficiency of the study and provide control.

1 Following all of these procedures, the supernatants were removed from the wells and were retained for 10 minutes with 100 μl cold methanol.
The plates which were ready for the ELISA process were then worked.

The following steps have been applied for the ELISA Test:
The supernatants of the present plate were emptied;
The plate was washed three times with 200 μl PBS-T20;
Toxoplasma gondii antibody IgG was diluted in PBS-%1 BSA solution at a ratio of 1/100;
Primary antibody was loaded as 100 μl and was incubated for 120 minutes;
The sample was washed three times with 200 μl PBS-T20;
Anti-Rabbit IgG (Alkaline Phosphatase) secondary antibody; was washed with PBS—1% BSA-%0.05 T20 and was diluted at a ratio of 1/1000;
Secondary antibody was loaded as 100 μl and was incubated for 75 minutes;
The reaction product was washed three times with 200 μl PBS-T20;
p-nitrophenyl phosphate substrate buffer (inside 0.1 M Tris Base, 0.1 M NaCl and 5 mM MgCl₂) was dissolved as 1 mg/ml;
100 μl pNPP each was loaded and incubated for 45 minutes being protected from light;
Stop solution (KOH 3N)50 μl was added.

The result was read by the ELISA reader device in 30 minutes.

48 hour efficiency: While pyrimethamine has an OD value of 45% at 4 μg/ml dose according to positive control (PK), reproduction of respectively 56%, 54% and 75% was observed at 2.1 and 0.5 μg/ml doses. Sulphadiazine in comparison to PK was determined as 51% at a concentration of 100 μg/ml, 97% at 50 μg/ml, 80% at 25 μg/ml and respectively as 79%, 61%, 95%, 84% and 97%.

Pyrimethamine had values of48%, 29%, 36%, 41%, 32%, 27%, 27%, 73% starting from 4 μg/ml respectively according to doses in 72 hour plates. Sulphadiazine was determined to have the values of 52%, 57%, 52%, 77%, 75%, 55%, 52%, 84% starting from 100 μg/ml.

The 48 hour values of Ginger (Zingiber officinale) in comparison to the control group was found to be respectively 38%, 64%, 63%, 79%, 79%, 100%, 97%, 100% according to 800 μg/ml and its half-half dilutions. The 72 hour values were determined to have reproduction values 21%, 39%, 64%, 72%, 80%, 99%, 94%, 100% respectively.

The DMSO content of 1600 μg/ml, 3200 μg/ml and above doses were around 0.1-2% and the ELISA values at 48 and 72 hours were determined to be 30% and 25% at 1600 μg/ml and 25% and 18% respectively at 3200 μg/ml.

It has been noted that the vegetal extraction and the solvent content (emulsion) that has been formed was effective on Gram positive and gram negative microorganisms and fungi following testing and it has also been discovered that it was effective in treating and preventing the reproduction of Staphylococcus spp., Enterococcus spp., Escherichia coli, Pseudomonas spp., Acinetobacter spp., Klebsiella spp. Etc. It especially has efficiency in comparison to the referred antimicrobial agents in agar penetration and MIC tests that have been carried out by taking the zone sizes and MIC (Minimum Inhibitory Concentration) values that have been described in the CLSI (The Clinical and Laboratory Standards Institute) guides.

As a result of the findings and the results obtained above, it has been determined that the invention can be applied to all kinds of fields of the industry primarily the human health and medicine sector.

What is claimed is:

1. A method of treating cancer, parasite, microbial or fungal disease, comprising:
administering a Ginger (Zingiber officinale) extract solution to treat the cancer, parasite, microbial or fungal disease;
wherein the Ginger (Zingiber officinale) extract solution comprises extracts of Ginger (Zingiber officinale), and a DMSO (dimethyl sulphoxide) solvent;
and wherein the extracts of Ginger (Zingiber officinale) are obtained by cold pressing; and an effective dose of the extracts of Ginger (Zingiber officinale) is between a range of 25 μg/ml-12800 μg/ml, and a content of the DMSO (dimethyl sulphoxide) solvent is between a range of 0.1-2 wt %;
and an anti-parasite effective dose of the extracts of Ginger (Zingiber officinale) on Toxoplasma gondii is 50 μg/ml-3200 μg/ml, and the content of the DMSO (dimethyl sulphoxide) solvent is 0.1 wt %.

2. The method of treating cancer, parasite, microbial or fungal disease according to claim 1, wherein a dose of 50 μg/ml-1600 μg/ml of the extracts of the Ginger (Zingiber officinale) is dissolved with the DMSO (dimethyl sulphoxide) solvent, and the content of the DMSO (dimethyl sulphoxide) solvent is 0.1 wt %.

3. The method of treating cancer, parasite, microbial or fungal disease according to claim 1, wherein an EC₅₀ (effective concentration) dose of the extracts of Ginger (Zingiber officinale) on cancerous (neoplastic) cells is 400 μg/ml-1600 μg/ml.

4. The method of treating cancer, parasite, microbial or fungal disease according to claim 3, wherein the EC₅₀ (effective concentration) dose of the extracts of Ginger (Zingiber officinale) on laryngeal cancer cells is 800 μg/ml.

5. The method of treating cancer, parasite, microbial or fungal disease according to claim 1, wherein the Ginger (Zingiber officinale) extract solution has an anti-parasite activity, and the anti-parasite effective dose of the extracts of Ginger (Zingiber officinale) is 800 μg/ml, 400 μg/ml, 200 μg/ml, 100 μg/ml, or 50 μg/ml.

6. The method of treating cancer, parasite, microbial or fungal disease according to claim 1, wherein the Ginger (Zingiber officinale) extract solution having an antimicrobial activity has a treatment effect and a reproduction preventive effect on Gram positive and Gram negative microorganisms, and wherein the Gram positive and Gram negative microorganisms comprise Staphylococcus spp., Enterococcus spp., Escherichia coli, Pseudomonas spp., Acinetobacter spp., and Klebsiella spp.

7. The method of treating cancer, parasite, microbial or fungal disease according to claim 2, wherein an EC₅₀ (effective concentration) dose of the extracts of Ginger (Zingiber officinale) on cancerous (neoplastic) cells is 400 μg/ml-1600 μg/ml.

8. The method of treating cancer, parasite, microbial or fungal disease according to claim 7, wherein the EC₅₀ (effective concentration) dose of the extracts of Ginger (Zingiber officinale) on laryngeal cancer cells is 800 μg/ml.

9. The method of treating cancer, parasite, microbial or fungal disease according to claim 2, wherein the Ginger (Zingiber officinale) extract solution has an anti-parasite activity, and the anti-parasite effective dose of the extracts of Ginger is 800 μg/ml, 400 μg/ml, 200 μg/ml, 100 μg/ml, or 50 μg/ml.

10. The method of treating cancer, parasite, microbial or fungal disease according to claim 2, wherein the Ginger (Zingiber officinale) extract solution having an antimicrobial activity has a treatment effect and a reproduction preventive effect on Gram positive and Gram negative microorganisms, and wherein the Gram positive and Gram negative microorganisms comprise Staphylococcus spp., Enterococcus spp., Escherichia coli, Pseudomonas spp., Acinetobacter spp., and Klebsiella spp.
